(19) United States
(12) Patent Application Publication
(10) Pub. No.: US 2007/0112336 A1
(43) Pub. Date: May 17, 2007

(54) Protective Cover for Endoscopic Tool

(75) Inventors: Amram AIZENFELD, Menashe (IL); Golan SALMAN, Atlit (IL); Yakov Bar Or, Haifa (IL)

Correspondence Address: BRUCE E. LILLING;LILLING & LILLING PLLC, P.O. BOX 560, GOLDEN BRIDGE, NY, 10526, US

(73) Assignee: Stryker GI Ltd., Haifa (IL)

(21) Appl. No.: 11/560,467

(22) Filed: Nov. 16, 2006

Related U.S. Application Data
(60) Provisional application No. 60/738,002, filed on Nov. 17, 2005.

(51) Int. Cl. A61B 17/00

(52) U.S. Cl. 606/1

(57) ABSTRACT

A cover for protecting a flexible shaft of an endoscopic tool from contamination and from the environment is disclosed. The cover comprises a storing portion for receiving the shaft of the endoscopic tool and a proboscis portion, which is connectable to the storing portion so as to be in flow communication therewith and to define a passage for the tool shaft when it is being protracted from the storing portion or retracted in the storing portion.

### BACKGROUND OF THE INVENTION

1. Field of the Invention

The present invention relates generally to endoscopy, and specifically to a protective cover for sheathing an endoscopic tool after its withdrawal from a body passage.

The present invention refers also to a protective cover assembly and to an endoscopic system, which employs such protecting cover.

2. Summary of the Related Art

The use of a disposable sleeve (also referred to as a sheath) to cover an endoscope is well known in the art. Flexible endoscopes, such as colonoscopes, are notoriously difficult to clean and disinfect thoroughly, leading to problems of cross-contamination between patients. These problems can be avoided by covering the endoscope with a single-use sleeve, which is discarded after use.

Endoscopes commonly have working channels, running from a proximal port outside the body to a distal port at the distal end of the endoscope. When the distal end of the endoscope is inserted into the body, the working channel may be used, inter alia, to pass a surgical instrument through to the distal end of the endoscope in order to perform a surgical procedure, such as a biopsy. The working channel serves also for supplying vacuum, when suction is required. Instruments that are used in this manner become contaminated with biological matter from inside the patient's body. As the instrument is withdrawn from the body, it can spread the contamination from the interior of the working channel to the proximal tool port of the endoscope and to the hands of an operator.

Silverstein (U.S. Pat. No. 5,695,491) discloses a containment system for containment of at least a major portion of the shaft of the working tool. However, this containment system is adapted to contain the shaft remote from the endoscope and it is not designed to cover that portion of the shaft, which is between the containment container and the endoscope.

Methods for sheathing an endoscope while providing working channels that protect the endoscope from contamination are described, for example, in Silverstein (U.S. Pat. No. 4,646,722) and Sidall (U.S. Pat. No. 4,741,326), whose disclosures are incorporated herein by reference. These patents attempt to prevent contamination of the endoscope, either by adding disposable working channels external to the endoscope itself (Silverstein—U.S. Pat. No. 4,646,722) or by adding a disposable liner inside a working channel of the endoscope (Sidall—U.S. Pat. No. 4,741,326). They do not address the problem, however, of contamination that may be spread to the area around the proximal end of the endoscope and to the operator's hands as the surgical tool is retracted from the proximal port of the working channel.

An attempt to solve this problem is described for example in Aizenfeld (U.S. Pat. No. 6,908,428), which is incorporated herein by reference. This patent discloses methods and devices for use in sheathing an endoscopic tool as it is removed from the patient's body. Such sheathing prevents contaminants that may adhere to the tool inside the body from contacting the operator' s hands, the handle of the endoscope, or other objects outside the patient's body. As a result, the likelihood of cross-contamination between patients is reduced, and the job of cleaning and disinfecting the endoscope and ancillary equipment between uses is simplified.

In embodiments of this solution, a sheathing assembly is provided, which comprises a sleeve dispenser mating with the proximal port of an endoscopic working channel, outside the patient's body. A flexible sleeve is typically fixed by its distal end to the dispenser, with the remainder of the sleeve bunched inside or otherwise held in a vicinity of the dispenser. An elongate endoscopic tool is passed through the dispenser and the working channel, until the distal end of the tool protrudes from the distal end of the endoscope. While the shaft of the tool is advanced through the dispenser and the proximal port of the working channel, the sleeve remains bunched at the dispenser. When the tool is retracted, however, the proximal end of the sleeve engages the shaft of the tool, so that, as the tool is withdrawn, the sleeve unfurls from the dispenser to cover the shaft of the tool, up to and including its distal end. All contaminants on the tool thus remain within the sleeve, while the outside of the sleeve remains clean and can be handled freely without spreading contamination.

Unfortunately this solution suffers from the fact that, when the tool is being retracted from the working channel, there exists a possibility that the distal end of the sleeve might be inadvertently torn and detached from the location where it is anchored to the sheathing assembly. Accordingly the tool might become exposed and contaminations adhered to the tool would spread to the environment.

Another disadvantage of the above solution lies in the fact that after the tool has been already retracted from the working channel and is protracted therein again, the sleeve bunches immediate before the sheathing assembly and impedes the doctor's fingers to grasp the tool as close as possible to the proximal port as would be desirable for easy and sure advancement of the tool inside the port.

Moreover, since the tool shaft deflects within the bunched portion this opposes the advancement of the tool and renders it difficult.

The present invention seeks to eliminate the above-mentioned disadvantages of the known solution.

### BRIEF DESCRIPTION OF THE SEVERAL VIEWS OF THE DRAWINGS

FIG. 1 is a schematic, pictorial illustration of a system for performing an endoscopic procedure, in accordance with an embodiment of the present invention;

FIG. 2a is a schematic, sectional view of a protecting cover in accordance with an embodiment of the present invention;

FIG. 2b is an enlarged view of the distal extremity of the proboscis portion.

FIGS. 3a is a schematic, pictorial illustrations showing how an endoscopic tool passes within the protective cover in accordance with various embodiments of the present invention;

FIG. 3b is similar to FIG. 3a, but there is a bag with a longitudinal fold;

FIG. 4 depicts a “milking” movement in a forward direction, which should be carried out for displacement of the endoscopic tool through the protecting cover;

FIG. 5 depicts a “milking” movement in a backward direction, which should be carried out for displacement of the endoscopic tool through the protecting cover;

FIG. 6 shows how the tool shaft deflects and loops within the protecting cover;

FIG. 7 is a view similar to FIG. 6, but shows the tool shaft with loops and figure eights within the protecting cover;

FIG. 8a shows transfer of a tissue sample to a sample container where the tool has been retracted;

FIG. 8b is a view similar to FIG. 8a, but the tool is within the container;

FIG. 9a depicts an embodiment of a protecting assembly comprising protective cover of the invention with an internal guiding tube within the bag;

FIG. 9b is a view similar to FIG. 2a, but showing an internal guiding tube within the bag; and

FIG. 9c is a view showing the exit end of the storing portion and the proboscis portion.

### DETAILED DESCRIPTION OF THE PREFERRED EMBODIMENT OF THE INVENTION

The present invention will be more fully understood from the following detailed description of the embodiments thereof, taken together with the drawings.

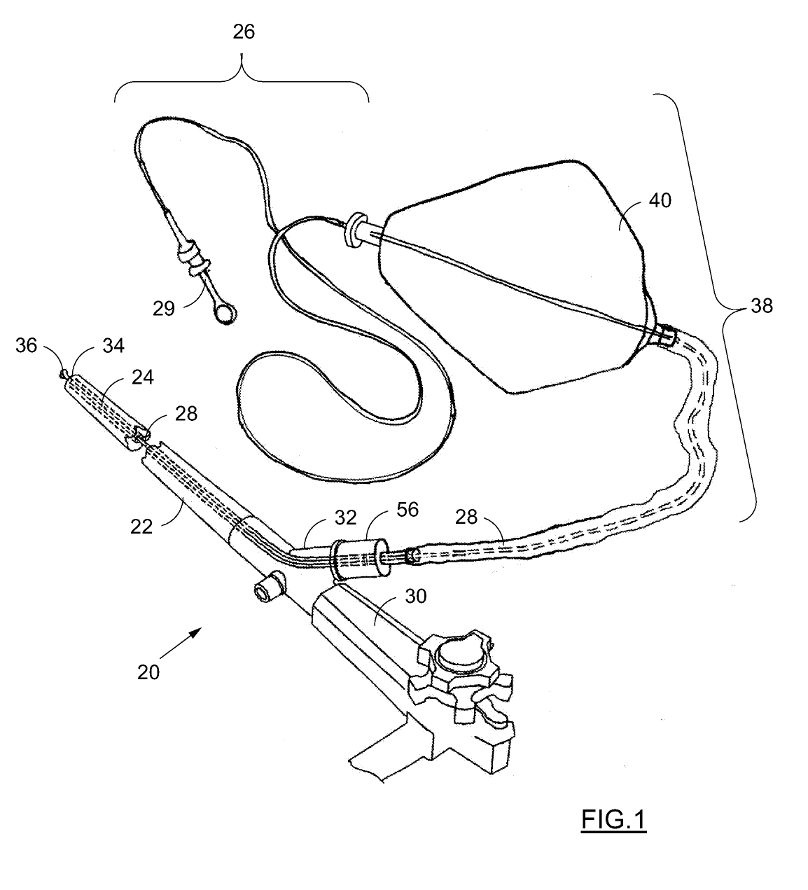

FIG. 1 is a schematic, pictorial illustration of an endoscopic system 20 for performing an endoscopic procedure, in accordance with an embodiment of the present invention. System 20 comprises an endoscope 22, having a working channel 24 passing therethrough. Channel 24 passes through endoscope 22 from a proximal port 32, typically in or near an operating handle 30 of the endoscope, to a distal port 34 at the distal end of the endoscope. An endoscopic tool 26 is inserted through channel 24 in order to access an area adjacent to the distal end of the endoscope, within the patient's body. Typically, the endoscopic tool 26 comprises an elongate shaft 28, with a working element 36 at its distal end and with a working handle 29 at its proximal end, as is known in the art. In the example shown in FIG. 1, working element 36 comprises biopsy forceps, which are operable to take a tissue sample within the patient's body, adjacent to distal port 34. Alternatively or additionally, channel 24 may be used to apply suction to a body passage through distal port 34 or to apply liquid or gas to the area outside the distal port, as is likewise known in the art.

A disposable sheath may cover insertion tube of endoscope 22, and channel 24 may likewise be internally sheathed, in order to protect the endoscope from contamination, e.g. as described in Sidall (U.S. Pat. No. 4,741,326). Alternatively or additionally, although channel 24 is shown in the figure as passing inside the endoscope, the endoscopic working channel may comprise a separate tube, typically disposable, which is fixed alongside the endoscope, as described, for example, in Silverstein (U.S. Pat. No. 4,646,722). The present invention is suited for use with either of these types of working channels.

Since at least working element 36 of tool 26 comes into contact with tissue and other biological matter inside the patient's body, the tool and the interior of the working channel (or the internal sheath which provides lining for the working channel) necessarily become contaminated during use. Furthermore, the interior of the working channel could be contaminated during suction and accordingly the tool shaft will be also contaminated when it passes through a contaminated working channel.

In order to prevent the spread of contamination from tool 26 to operating handle 30, to the operator's hands and to other areas outside the patient's body, a protective cover 38 is attached to port 32. Protective cover 38 is connected to port 32 so that a passage through the cover is aligned with working channel 24. Tool 26 is then inserted through the cover into working channel 24 and can be used in the usual manner.

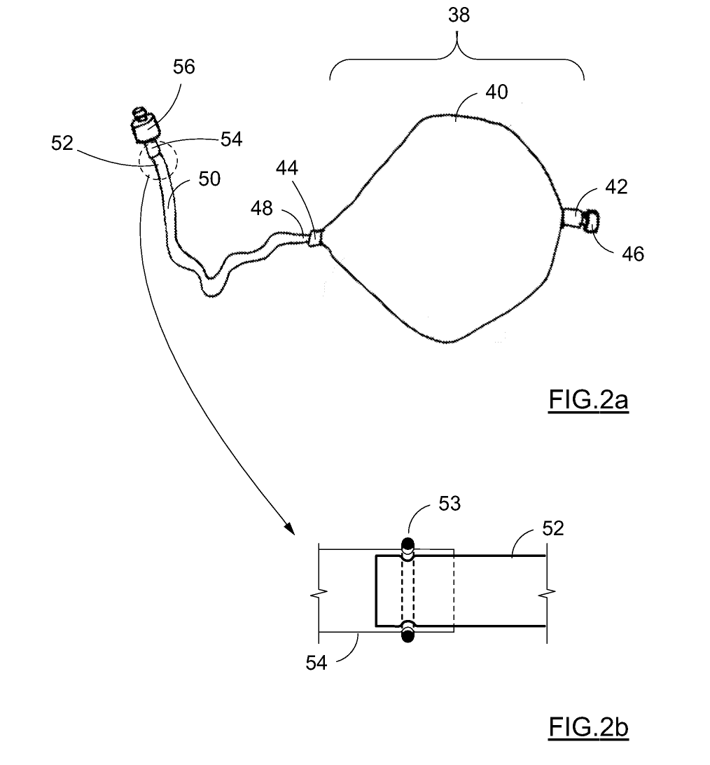

FIG. 2 is a schematic view of protective cover 38, in accordance with an embodiment of the present invention. The protective cover is shown without the tool while being ready to receive it.

In the present embodiment, protective cover 38 comprises its main element, which is a storing portion 40 adapted for hosting therein shaft 28 of the tool during protracting thereof into or retracting from proximal port 32. By virtue of this provision the tool is never exposed to the environment and any spreading of contamination therefrom is prevented.

The storing portion is configured as a flat, relatively wide, plastic bag, which has a proximal, entrance end 42 and a distal, exit end 44. In practice the bag can be made of a high density polyethylene or other suitable plastic material and it has wall thickness of about 0.02-0.03 mm.

The tool can enter the bag through the proximal end and exit from the bag through the distal end. In practice the distal end can be temporarily closed by a disposable resilient plug to prevent the early exit of the tool shaft from the distal end.

The entrance end 42 of the bag is provided with a one-way plug 46, which ensures that the tool shaft can be only protracted through the entrance end, while its retraction from the bag is prevented. Instead of the one-way plug one can use a means suitable for securing the rear part of the tool shaft at the entrance end and allowing only protracting of the tool shaft in the storing portion. An example of such a means could be a collet, similar to that used for holding cylindrical pieces in a lathe, or it could be an eccentric fixture.

The further component of the protective cover is a proboscis portion 50, which is coupled to the exit end 44 of the storing portion.

It is seen that to the exit end 44 of the bag is coupled a proximal end 48 of the proboscis portion 50. The proboscis portion is configured as a sleeve made from easily pliable plastic material. A distal end 52 of the proboscis portion is anchored to an adapter 54, which, in its turn, terminates by a connecting fitting 56 attachable to proximal port 32.

Proximal extremity 48 of the proboscis portion is connected by an appropriate connection to distal end 44 of the storing portion. The distal extremity 52 of the proboscis portion is anchored to adapter 54, e.g. by an elastic ring 53 put over a groove made in the adapter. This arrangement is schematically depicted on enlarged fragment of FIG. 2. Alternatively the anchoring can be accomplished by a conical ring put over the distal extremity so as to be in snapping relationship therewith.

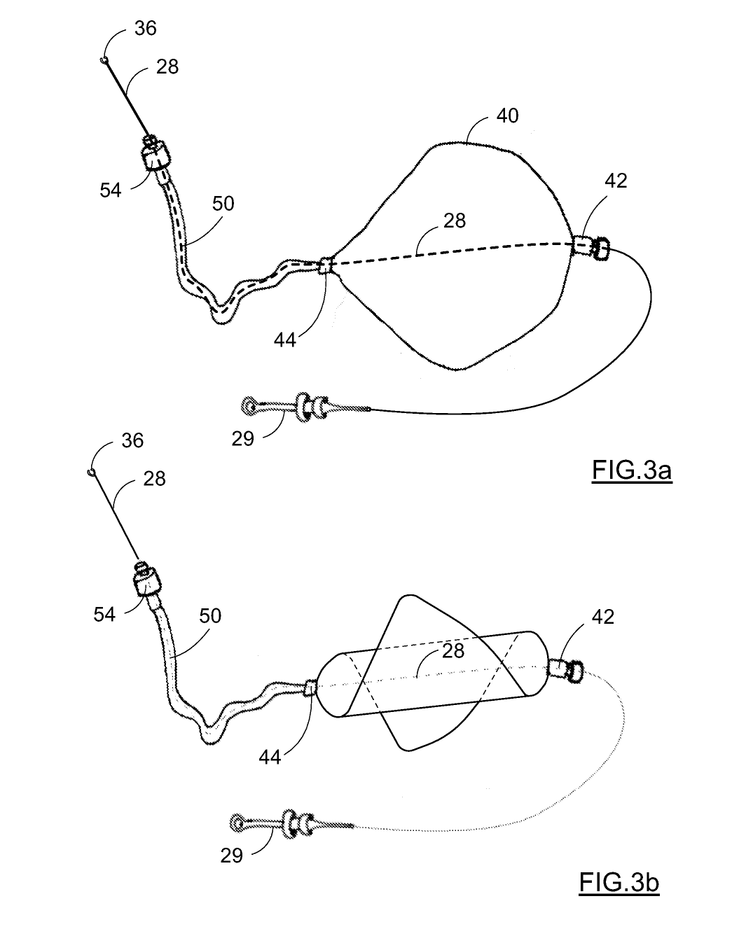

Referring now to FIG. 3a it is shown the situation when the tool has been inserted into storing portion 40 through its proximal, entry end 42 and then advanced in the forward direction until distal end of the tool shaft with working element 36 has passed the exit end 44, the proboscis portion 50 and protrudes outside the cover assembly through adapter 54. The proboscis portion is made of a pliable plastic material and is preferably provided with corrugations or folds to enable easy and convenient squeezing by fingers. At the same time the material of the proboscis portion should provide good friction between the tool shaft and the proboscis portion. Examples of suitable plastic materials are polyurethane, polyethylene, etc. The wall thickness of the proboscis portion should be selected to permit easy squeezing and reliable grasping the tool shaft by fingers. In practice the wall thickness is about 0.5 mm.

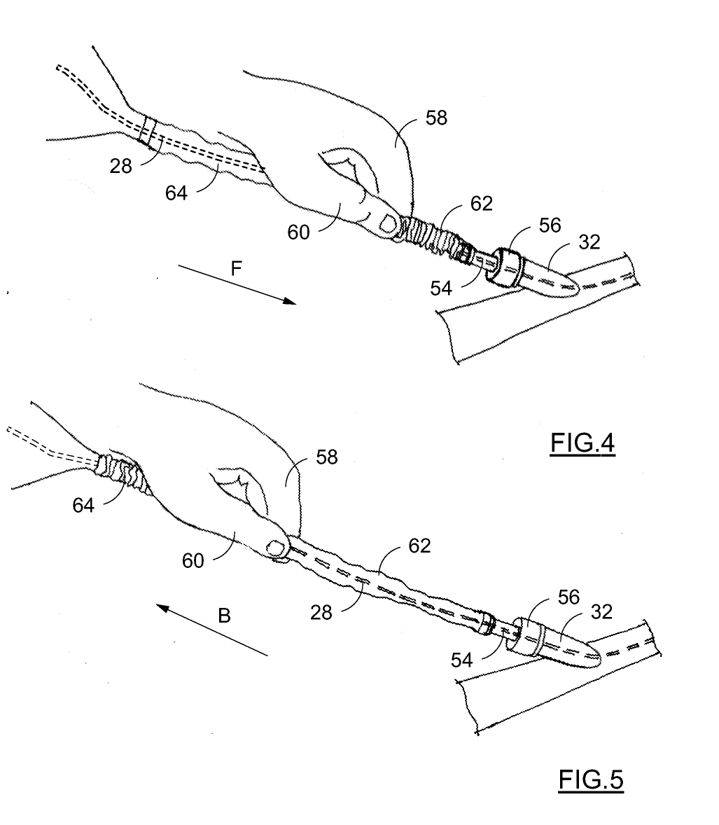

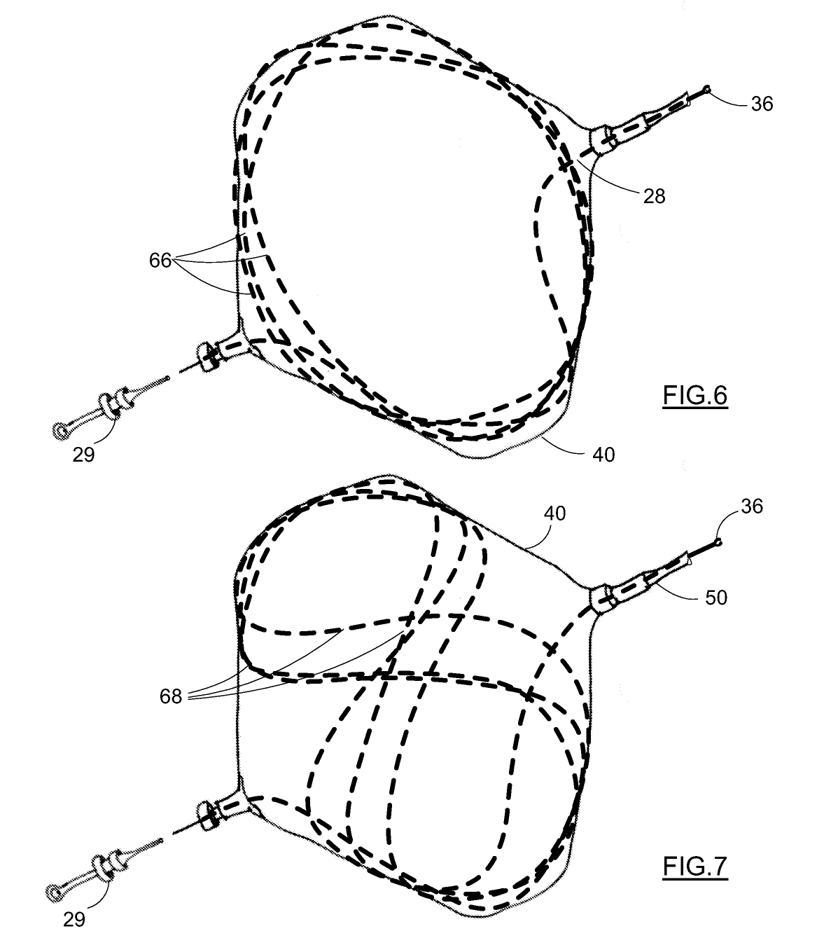

Once adapter 54 is connected by fitting 52 to port 32, the endoscopic tool can be protracted into the working channel, e.g. for taking a biopsy sample. Then the tool shaft can be retracted from the proximal port, the adapter can be disconnected from the proximal port and the biopsy sample can be transferred in a dedicated sample container. For protracting or retracting of the tool shaft through the storing portion one should grasp the tool shaft by fingers in an initial position close to the connecting fitting, as shown in FIGS. 4 and 5, and then to displace the tool shaft forward or backward while squeezing the corrugations of the proboscis portion. With reference to FIGS. 4 and 5 it is shown how doctor's fingers 58, 60 initiate squeezing of the proboscis portion while grasping the tool shaft. Then the shaft is displaced in the forward or backward direction as designated by respective arrows F,B. The tool shaft is displaced by virtue of a “milking” movement, which comprises repetitive advancing the shaft forward or backward from the initial position, then release the shaft in a new position, while still grasping the proboscis portion, and then returning the fingers in the initial position along with the proboscis portion.

In the prior art endoscopes, which do not employ protecting covers, the doctors are familiar with protracting the tool into the port and retracting it from the port by the same “milking movement” and therefore they should not change their habit while using the protective cover of the present invention.

It can be readily appreciated that, when the doctor's fingers advance the tool shaft forward to protract it into the port, the corrugations of the proboscis portion bunch between the initial position and the adapter. This situation is depicted in FIG. 4, in which the bunched region is designated by reference numeral 62. The region of the proboscis portion behind the fingers is designated by reference numeral 64. When the region 62 bunches, the region 64 straightens up. At the same time when the shaft is advanced backward, i.e. is retracted from the port, region 64 bunches and region 62 straightens up. It should be borne in mind, however, that, when either the region 62 or 64 bunches, it nevertheless does not provide a hindrance for protracting or retracting the tool shaft through the proboscis portion and the doctor's fingers can always remain close to the proximal port of the working channel. By virtue of this provision the advancement of the tool shaft through the port is easy, reliable and efficient.

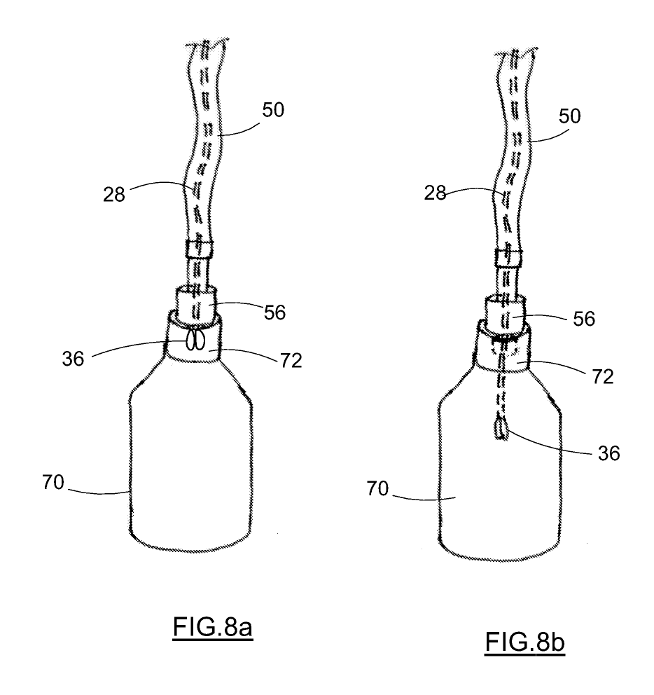

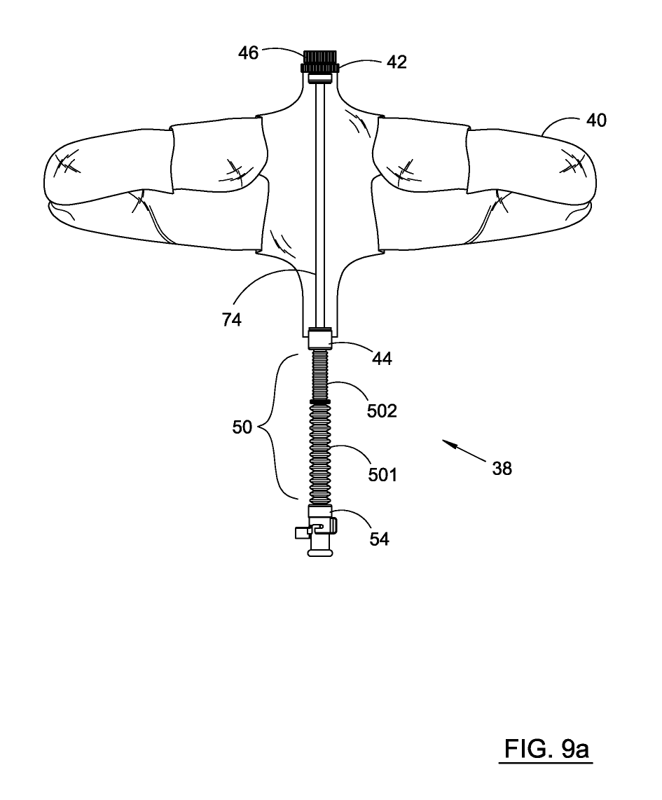

Attention is now called to FIGS. 6 and 7, which show how the tool shaft 28 is gathered in the storing portion 40 and is stored therein being sheathed and thus prevented from exposure to the environment. In accordance with the present invention the storing portion is configured and dimensioned in such a manner that when the tool shaft retracts from proximal port 32 of the endoscope, the interior of the storing portion urges the tool shaft to elastically deflect and make loops 66 inside the storing portion. By virtue of this provision the tool shaft is compactly stored. Depending on the bag's size and configuration the tool shaft can deflect by making loops and even figure eights 68. This situation is seen in FIG. 7 depicting the shaft gathered in a compact configuration consisting of loops and figure eights.

In practice the protective cover of the invention is used as follows. First, the preparation step is carried out during which the tool shaft is entered into proximal end 42 of the storing portion and then pushed towards distal end 44 through proboscis portion 50 until working element 26 of the tool slightly protrudes outside from fitting 56. For carrying this out distal end 42 of the storing portion should be aligned with its proximal end 44. To make the alignment procedure easier it is advantageous to provide the bag with a longitudinal fold extending between distal end 42 and proximal end 44. This situation is shown in FIG. 3b.

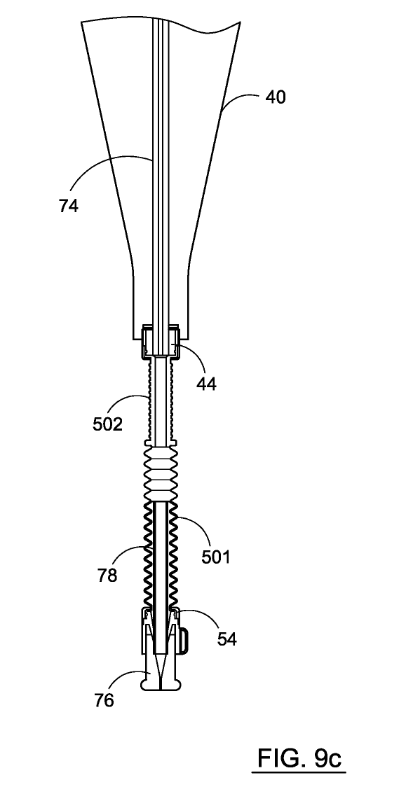

In an embodiment of the invention a tubular guide can be provided within the storing portion for aligning the distal and proximal end of the storing portion. The guide facilitates alignment between the distal end of the tool shaft and the proboscis portion and by virtue of this provision entering the tool shaft in the proximal end of the proboscis portion becomes easy and convenient. This embodiment will be explained in more details with reference to FIG. 9.

After inserting the tool in the storing portion the connection fitting 56 is attached to proximal port 32 of the endoscope and the tool is protracted thereinto until tool's operating handle 29 is in vicinity to proximal end 42 of the storing portion as shown in FIG. 3. In this position the rear portion of the tool shaft can be secured at the proximal end of the bag 40 (if instead of the one-way plug an alternative means for securing the tool is used).

After completing the preparation step the tool shaft can be either retracted from proximal port 32 or protracted thereinto by virtue of the above described “milking” movement applied to proboscis portion 50.

It might be advantageous if the proboscis portion is made of a material, which is easily pliable and at the same time is also elastically stretchable. An example of a suitable material can be silicon or polyurethane. Since elastically stretchable proboscis portion would act as a spring, the probability for its detachment from adapter 52 is much less.

Up to now an embodiment of the protecting cover assembly has been described, in which the protective cover constitutes an item, which is independent from the endoscopic tool. This item can be supplied separately from the tool and for its use during the endoscopic procedure one should complete the above described preparation step.

However, one can contemplate also a situation, in which the protective cover assembly and the tool are supplied as a protective cover assembly, i.e. as a ready for use combination, in which the endoscopic tool had been already deployed within the storing portion. In such protective cover assembly there is no need in preparation step and the endoscopic tool is ready for protracting into proximal port 32 of an endoscope upon connection fitting 56 to the proximal port

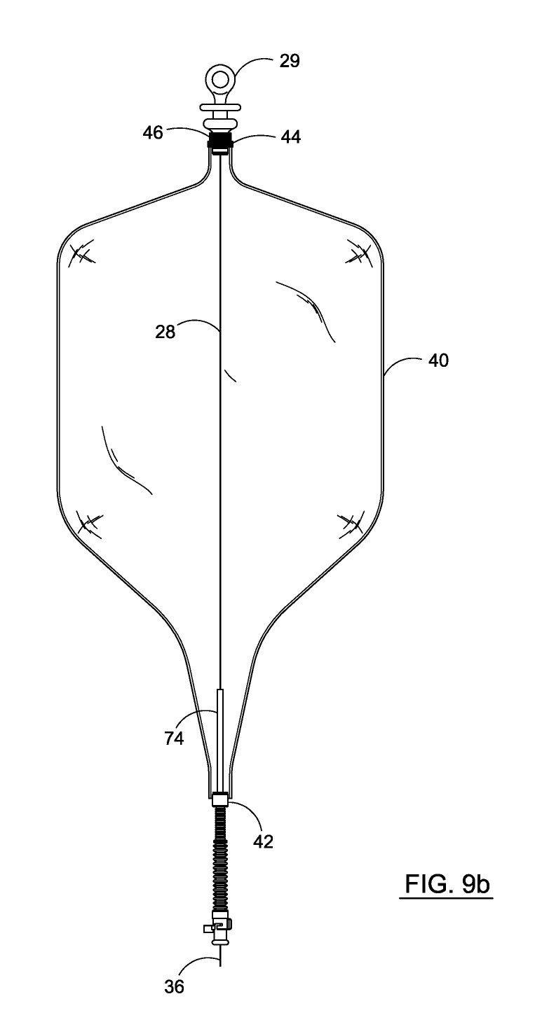

FIGS. 8a and 8b provide a schematic, pictorial illustration showing transfer of a tissue sample from working element 36 of the endoscopic tool to a sample container 70. In FIG. 8a is seen the tool when it has been retracted completely from the endoscope, and adapter 54 has been detached from port 32. Then adapter 54 is coupled preferably by the same fitting 56 to a neck 72 of container 70, as shown in FIG. 8b. At this point, assuming working element 36 to comprise biopsy forceps holding a biopsy sample captured inside the patient's body, for example, the operator may advance working element 36 into container 70 through neck 72, and may then open the forceps, releasing the biopsy sample into the container. Thus, working element 36 and the sample that it captures are never exposed to the environment outside storing portion 40, proboscis portion 50 and container 70.

Now with reference to FIG. 9 an embodiment of the protecting assembly will be explained, in which the storing portion is provided with an internal guiding tube.

Since the protecting assembly of this embodiment has the same main components as the assembly explained previously in connection with FIG. 3a, the similar components are designated by the same reference numerals. Among the similar components are storing portion 40 provided with respective entrance and exit end 42, 44 as well as the proboscis portion 50 provided with adapter 54. As in the previous embodiment the storing portion comprises a plastic bag, which is shown in FIG. 9a as being crumpled in a compact state such that the distance between the entrance end and the exit end can be shortened. Within the bag there is provided an inner guiding tube 74, which bridges between the entrance end and the exit end and thus brings them in alignment with a possibility for fluid communication therebetween. The proximal end of the guiding tube is detachably connected to the entrance end 44 of the storing portion, while the distal end of the guiding tube is secured in the exit end 42 of the storing portion. The tube is made of a relatively rigid plastic material, e.g. PVC, and by virtue of this provision, as soon as the shaft is being protracted through the entrance end, it is guided within the guiding tube until it reaches the exit end and protrudes therefrom. During protracting of the tool, the proximal end of the guide tube detaches from the entrance end 44 and the storing portion unfolds from the crumpled state to the flat state. This situation is shown in FIG. 9b, in which it is seen that tool shaft 28 extends along the storing portion 40 and along the guiding tube 74. Distal end 36 of the tool protrudes from the storing portion and proximal end of the tool is secured by a fixture 46 at the entrance end 44 of the storing portion. Proximal end of the guiding tube is detached from the exit end of the storing portion. The storing portion is flat and not crumpled.

By virtue of the guiding tube the shaft reaches the exit end and easily passes through the storing portion even in a situation when the storing portion is folded.

It might be advantageous if the proboscis portion consists of two sections, namely a long section 501 and a short section 502. Both sections are corrugated and the diameter of the long section is more than the diameter of the short section. During the “milking” movement the operator's fingers grasp the short section and squeeze the long section.

In FIG. 9c is shown the exit end of the storing portion and the proboscis portion. A disposable stopper plug 76 is provided at the distal end of the proboscis portion. The plug is made of a resiliently deformable material and is detachably connectable to adapter 54. The plug is retained in place during the preparation step when the tool is being advanced along the storing portion until the distal end thereof reaches the distal end of the proboscis portion. To make the advancement easier, an auxiliary guiding tube 78 can be provided within the proboscis portion. When the tool reaches the distal end, the plug is taken out from the adapter and thus the storing assembly can be connected to the port of the endoscope and the tool can be protracted thereinto.

Protective cover of the invention is particularly suited for use with flexible endoscopes that are inserted into the gastrointestinal tract, such as colonoscopes and gastroscopes. Alternatively, the principles of the present invention may be applied to sheath tools that are inserted through lumens in medical probes of other types, such as endoscopes (both rigid and flexible) and catheters used in other body passages and in other therapeutic and diagnostic procedures.

One should bear in mind that the storing portion need not necessarily be configured as a flat bag, i.e. as a substantially two dimensional receptacle. One can contemplate an embodiment in which the storing portion is configured as a ball for rugby, i.e. as a three dimensional receptacle.

It will thus be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

We claim:

1. A cover for protecting a flexible shaft of an endoscopic tool, said cover comprising
a storing portion for receiving the flexible shaft of the endoscopic tool, said storing portion being configured as a pliable receptacle having an proximal end through which the flexible shaft can be protracted within the storing portion and a distal end through which the flexible shaft can be either protracted from the storing portion to a port of said endoscopic tool or retracted from the port of the endoscopic tool back into the storing portion, said storing portion being configured to urge the tool shaft to elastically bend within the storing portion when the shaft is being protracted into the storing portion through the proximal end or retracted into the storing portion through the distal end;
said cover further comprising
a proboscis portion, which is connectable to the storing portion to be in flow communication therewith and to define a passage for the flexible shaft when it is being protracted from the storing portion or retracted into the storing portion, said proboscis portion comprising a proximal end, which is fixed to the distal end of the storing portion, and a distal end, which is detachably connectable to the port of the endoscopic tool.

2. The cover as defined in claim 1, wherein said storing portion being substantially flat.

3. The cover as defined in claim 2, wherein said storing portion being configured as a plastic bag.

4. The cover as defined in claim 1, wherein the proximal end of said storing portion being provided with a securing means to allow a rear part of the flexible tool to be only protracted through the proximal end, while retraction through the proximal end from the storing portion is prevented.

5. The cover as define in claim 4, wherein said securing means being a one-way plug.

6. The cover as defined in claim 5, wherein the distal end of said proboscis portion being provided with an adapter for connecting to the port of the endoscope.

7. The cover as defined in claim 1, wherein said storing portion being provided with a guiding tube for aligning the proximal end of the storing portion with the distal end of the storing portion.

8. The cover as defined in claim 7, wherein a first end of the guiding tube being detachably connected to the proximal end of the storing portion and wherein a second end of the guiding tube being fixed to the distal end of the storing portion.

9. The cover as defined in claim 1, wherein either said storing portion or said proboscis portion being made of a plastic material.

10. The cover as defined in claim 1, wherein said storing portion and said proboscis portion being made of a plastic material.

11. The cover as defined in claim 1, wherein at least a section of the proboscis portion being provided with corrugations.

12. The cover as defined in claim 1, wherein the distal end of the proboscis portion being temporarily closeable by a plug.

13. A protective cover assembly comprising
an endoscopic tool provided with a flexible shaft and
a protective cover for protecting at least the flexible shaft of the endoscopic tool, wherein said protecting cover comprises
a storing portion for receiving the flexible shaft of the endoscopic tool, said storing portion being configured as a pliable receptacle having a proximal end through which the flexible shaft can be protracted within the storing portion and a distal end through which the flexible shaft can be either protracted from the storing portion to a port of said endoscopic tool or retracted from the port of said endoscopic tool back into the storing portion, said storing portion urging the flexible shaft to elastically bend within the storing portion when the flexible shaft is being protracted into the storing portion through the proximal end or retracted into the storing portion through the distal end, said protecting cover further comprises
a proboscis portion, which is connectable to the storing portion to define a passage for the flexible shaft when it is protracted from the storing portion or retracted into the storing portion, said proboscis portion comprising a proximal end, which is fixed to the distal end of the storing portion, and a distal end, which is detachably connectable to the port of the endoscopic tool.

14. The protective cover assembly as defined in claim 13, wherein said storing portion is substantially flat.

15. The protective cover assembly as defined in claim 13, wherein said storing portion is configured as a plastic bag.

16. The protective cover assembly as defined in claim 13, wherein the proximal end of said storing portion being provided with a securing means, which allows a rear part of the flexible shaft to be only protracted through the proximal end, while retraction through the proximal end from the storing portion is prevented.

17. The protective cover assembly as defined in claim 13, wherein the distal end of said proboscis portion being provided with an adapter for connecting to the port of the endoscopic tool.

18. The protective cover assembly as defined in claim 13, wherein said storing portion being provided with a guiding tube for guiding the flexible shaft from the proximal end of the storing portion to the distal end of the storing portion when the flexible shaft is being displaced within the storing portion.

19. An endoscopic system comprising
an endoscope fitted with a working channel passing therethrough from a proximal port situated near an operating handle to a distal port;
an endoscopic tool insertable through the working channel to access an area adjacent to the distal port of the endoscope, said endoscopic tool having a flexible shaft and a working element at a distal end thereof;
a protective cover for protecting at least the flexible shaft of the endoscopic tool, wherein said protective cover comprises a storing portion for receiving the flexible shaft of the endoscopic tool and a proboscis portion, said storing portion being configured as a pliable receptacle having a proximal end through which the flexible shaft can be protracted within the storing portion and a distal end through which the flexible shaft can be either protracted from the storing portion to the proximal port or retracted from the proximal port of the endoscopic tool back in the storing portion, said storing portion urging the flexible shaft to elastically bend within the storing portion when the flexible shaft is being protracted into the storing portion through the proximal end or retracted into the storing portion through the distal end, and said proboscis portion being connectable to the storing portion to define a passage for the flexible shaft when it is being protracted from the storing portion or retracted into the storing portion, said proboscis portion comprising a proximal end, which is fixed to the distal end of the storing portion, and a distal end, which is detachably connectable to the proximal port of the endoscopic tool.

20. The enodscopic system as defined in claim 19, wherein said distal end of said proboscis portion being provided with an adaptor for connecting to the proximal port of the endoscopic tool and said flexible shaft of the endoscopic tool being retractable from the proximal port or protractable thereinto through the protecting cover.
